(19) United States
(12) Patent Application Publication
(10) Pub. No.: US 2024/0383967 A1
(43) Pub. Date: Nov. 21, 2024

(54) HUMANIZED ANTIBODY INHIBITORS FOR CATHEPSIN S

(71) Applicant: UNIVERSITY OF SOUTHERN CALIFORNIA, Los Angeles, CA (US)

(72) Inventor: Yong ZHANG, Los Angeles, CA (US)

(21) Appl. No.: 18/666,067

(22) Filed: May 16, 2024

Related U.S. Application Data
(60) Provisional application No. 63/503,102, filed on May 18, 2023.

(51) Int. Cl. C07K 14/81; C07K 7/06; C07K 16/32

(52) U.S. Cl. CPC C07K 14/8139; C07K 7/06; C07K 16/32; C07K 2317/22; C07K 2317/55; C07K 2317/565; C07K 2317/76; C07K 2317/92; C07K 2319/73

(57) ABSTRACT

Embodiments of the invention include a fusion protein and/or cathepsin S inhibitors that comprise an antibody scaffold protein comprising a full-length antibody or a Fab fragment, and a propeptide of procathepsin S, such that the propeptide of procathepsin S is grafted into one or more complementarity determining regions (CDR) of the full-length antibody, or the propeptide of procathepsin S is grafted onto an N-terminus or C-terminus of a light chain and/or a heavy chain of the Fab fragment.

### RELATED APPLICATIONS

This application claims priority under 35 U.S.C. § 119(e) U.S. Provisional Patent Application No. 63/503,102, filed May 18, 2023, which is incorporated herein by reference.

### GOVERNMENT SUPPORT

This invention was made with government support under grant no. R35GM137901, awarded by the (NIH/NIGMS) National Institute of General Medical Sciences and grant no. R01EB031830, awarded by the (NIH/NBIB) National Institute of Biomedical Imaging and Bioengineering. The government has certain rights in the invention.

### SEQUENCE LISTING

The present application includes a Sequence Listing in electronic format as an xml file titled “530.035US1_SL” which was created on May 16, 2024, and has a size of 14,815 bytes. The contents of xml file 530.035US1_SL are incorporated herein by reference.

### BACKGROUND OF THE INVENTION

Cathepsin S (CTSS) is a cysteine protease. Unlike other members of the cathepsin family, CTSS remains catalytically active in a broad range of pH and features a relatively restricted expression profile. Phylogenetically, it belongs to the cathepsin-L like subfamily. CTSS plays important roles in antigen presentation mediated by major histocompatibility complex class II (MHC class II), matrix remodeling, and inflammation. Dysregulated CTSS expression and proteolytic activity are strongly implicated with many human diseases such as cancer, Alzheimer's disease, cardiovascular diseases, and autoimmune diseases. CTSS have thus emerged as an attractive therapeutic target.

To block the pathological enzymatic activity of CTSS, various types of small-molecule inhibitors against CTSS have been developed. None of them has yet to receive approvals for clinical use, likely due to limited efficacy and/or safety concerns. In comparison to small-molecule agents, monoclonal antibodies are characterized by high affinity and specificity. Moreover, the versatile immunoglobulin scaffolds enable incorporation of new functions and modulation of pharmacological properties. Antibody-derived inhibitors of CTSS may provide additional benefits in clinic. While anti-CTSS antibodies could be readily prepared, few antibodies with potent inhibition activity specific for CTSS are available.

There remains therefore a great need to develop safe, potent, high affinity inhibitors of CTSS. The current invention satisfies these needs.

### SUMMARY OF THE INVENTION

This disclosure relates to antibody inhibitors of CTSS made through genetic fusions of the propeptide of proCTSS into the heavy chain complementarity determining region 3 (CDR3H) of a full-length anti-human epidermal growth factor receptor 2 (HER2) antibody Herceptin (designated as Her-HC-CTSSpp IgG) or the N-terminus of the light chain of the fragment antigen-binding (Fab) of an anti-respiratory syncytial virus (RSV) F protein antibody Synagis (designated as Syn-LC-CTSSpp Fab) (FIG. 1). The resulting IgG and Fab fusion antibodies revealed selective inhibition activities toward CTSS at low nanomolar levels, representing new agents for anti-CTSS therapy.

Accordingly, embodiments of the invention include a fusion protein comprising an antibody scaffold protein that comprises a full-length antibody or a Fab fragment, and a propeptide of procathepsin S, wherein the propeptide of procathepsin S is grafted into one or more complementarity determining regions (CDR) of the full-length antibody, or the propeptide of procathepsin S is grafted onto an N-terminus or C-terminus of a light chain and/or a heavy chain of the Fab fragment.

In other embodiments, a humanized cathepsin S-antibody inhibitor comprises a fusion protein of Formula I: A-B-C-D-A, wherein: A is an antibody scaffold protein; B is a first coiled-coil domain; C is a propeptide of procathepsin S; and D is a second coiled-coil domain; wherein Formula I optionally includes one or more peptide linkers between A and B of segment A-B- and/or between D and A of segment -D-A.

The disclosure also provides for methods of treating a disease comprising administering an effective amount of the fusion protein, the cathepsin S inhibitor, or a composition comprising the fusion protein or the cathepsin S inhibitor to a subject in need thereof, thereby inhibiting cathepsin S and treating the disease. The disease can be one or more of cancer, Alzheimer's disease, a cardiovascular disease, and an autoimmune disease.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Rational design of humanized inhibitory antibodies for CTSS. (A) Generation of a full-length antibody inhibitor of CTSS (Her-HC-CTSSpp IgG) by grafting the propeptide of proCTSS into CDR3H of the anti-HER2 antibody Herceptin. (B) Synthesis of an antibody Fab inhibitor of CTSS (Syn-LC-CTSSpp Fab) by fusing the propeptide of proCTSS to the N-terminus of light chain of the Fab of the anti-RSV F protein antibody Synagis. The propeptide of proCTSS is in red (UniProt PDB ID NO: 2C0Y). The coiled coil-based stalk is bluc. Herceptin and Synagis immunoglobulin scaffolds are grey (PDB IDs: 1N8Z, 3D6G, and 2HWZ) (www.uniprot.org).

FIG. 2. Coomassie-stained SDS-PAGE gel analysis of purified antibodies. (A) Herceptin IgG. (B) Her-HC-CTSSpp IgG. (C) Synagis Fab. (D) Syn-LC-CTSSpp Fab.

FIG. 3A-F. Inhibition potency and specificity of Her-HC-CTSSpp IgG. (A) Inhibition activity of Her-HC-CTSSpp IgG for human CTSS. (B) Determined Kᵢ of Her-HC-CTSSpp IgG for human CTSS. (C) Inhibition activity of Her-HC-CTSSpp IgG for human CTSL. (D) Determined Kᵢ of Her-HC-CTSSpp IgG for human CTSL. (E) Effects of Her-HC-CTSSpp IgG on human CTSB proteolytic activity. (F) Effects of Her-HC-CTSSpp IgG on bovine trypsin proteolytic activity. Her-HC-CTSSpp IgG at various concentrations were incubated with human CTSS, CTSL, CTSB, or bovine trypsin in the presence of their respective fluorogenic substrates for up to 20 minutes. Proteolytic activities were monitored based on measured real-time fluorescence intensities. PBS and Herceptin were included as controls.

FIG. 4A-F. Inhibition potency and specificity of Syn-LC-CTSSpp Fab. (A) Inhibition activity of Syn-LC-CTSSpp Fab for human CTSS. (B) Determined Kᵢ of Syn-LC-CTSSpp Fab for human CTSS. (C) Inhibition activity of Syn-LC-CTSSpp Fab for human CTSL. (D) Determined Kᵢ of Syn-LC-CTSSpp Fab for human CTSL. (E) Effects of Syn-LC-CTSSpp Fab on human CTSB proteolytic activity. (F) Effects of Syn-LC-CTSSpp Fab on bovine trypsin protcolytic activity. Syn-LC-CTSSpp Fab at various concentrations were incubated with human CTSS, CTSL, CTSB, or bovine trypsin in the presence of their respective fluorogenic substrates for up to 20 minutes. Proteolytic activities were monitored based on measured real-time fluorescence intensities. PBS and Synagis were included as controls.

FIG. 5. Michaelis constants of fluorogenic substrates for human CTSS and CTSL. (A) Kₘ of Z-VVR-AMC for human CTSS. (B) Kₘ of Z-FR-AMC for human CTSL.

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012; also see the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moicty, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any clement, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, embodiment of the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CHI, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CHI, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The CDRs are primarily responsible for binding to an epitope of an antigen. The CDRs of cach chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. An antibody that binds HER2 or cathepsin B (also referred to herein as an anti-HER2 antibody or an anti-cathepsin B antibody) will have a specific VH region and the VL region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, “fragment” is defined as at least a portion of the variable region of the immunoglobulin molecule which binds to its target, i.e., the antigen binding region. Some of the constant regions of the immunoglobulin may be included.

As used herein, “fused” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct fusion involves one molecule attached to the molecule of interest. Indirect fusion involves one molecule attached to another molecule which in turn is attached directly or indirectly to the molecule of interest.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment disclosed herein that is a polynucleotide encompasses both the double-stranded form and each of two complementary single-stranded forms known or predicted to make up the double-stranded form.

As used herein, the terms “peptide,” “polypeptide,” and “protein” are used interchangeably, and refer to a compound comprising amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein, means a molecule, such as an antibody, which recognizes and binds to another molecule or feature, but does not substantially recognize or bind other molecules or features in a sample.

As used herein, the term “proCTSS” or “procathepsin S” are used interchangeably and refers to the protein identified as having Genbank accession no. AAC37592.1 (UniProt ID NO: 2C0Y) and having the amino acid sequence MKRLVCVLLVCSSAVAQLHKDPTLDHHWH LWKKTYGKQYKEKNEEAVRRLIWEKNLKFVMLHNLEHSMGMHSYDLGMNHLGD MTSEEVMSLMSSLRVPSQWQRNITYKSNPNRILPDSVDWREKGCVTEVKYQGSCGA CWAFSAVGALEAQLKLKTGKLVSLSAQNLVDCSTEKYGNKGCNGGFMTTAFQYIID NKGIDSDASYPYKAMDLKCQYDSKYRAATCSKYTELPYGREDVLKEAVANKGPVS VGVDARHPSFFLYRSGVYYEPSCTQNVNHGVLVVGYGDLNGKEYWLVKNSWGHN FGEEGYIRMARNKGNHCGIASFPSYPEI. (SEQ ID NO: 1).

As used herein, the term “cathepsin S” or “CTSS” is used interchangeably and means the mature active polypeptide comprising amino acids 115-331 from SEQ ID NO: 1.

As used herein, the term “CTSSpp” or “CTSS propeptide” or “propeptide of procathepsin S” are used interchangeably and refer to amino acids 17-113 of SEQ ID NO: 1.

As used herein, the term “effectively replaces” refers to a CDR domain, and in particular, a CDR3 domain, in which the binding activity of the CDR3 is eliminated by replacement of the CDR3 amino acid sequence with another amino acid sequence (e.g., the CDR3 is replaced by the propeptide of procathepsin S).

### Embodiments of the Invention

The disclosure generally provides enzymatic inhibitors and methods of using them, generally comprising, for example, a fusion protein comprising an antibody scaffold protein and a peptide-based enzymatic inhibitor of cathepsin S.

In some embodiments, the antibody scaffold protein comprises one or more of a polyclonal antibody, a monoclonal antibody, a chimeric antibody, a humanized antibody, an Fv fragment, a Fab fragment, an Fab′ fragment, an F(ab′)2 fragment, an Fab′-SH fragment, a single chain variable fragment (scFv), a single domain antibody, a diabody, a liner antibody, a bispecific antibody, and a multispecific antibody. In some embodiments, the antibody is Trastuzumab (Herceptin®) or another anti-HER2 antibody. In another embodiment, the antibody scaffold protein comprises one or more anti respiratory syncytial virus (anti-RSV Fab fragments. In some embodiments, the anti-RSV Fab fragment is Synagis® (palivizumab).

Antibodies (e.g., full length antibodies) used as the antibody scaffold protein generally comprise a variable region heavy chain and a variable region light chain. The antibodies may comprise derivatives or fragments or portions of antibodies that retain the antigen-binding specificity, and also preferably retain most or all of the affinity, of the parent antibody molecule. For example, derivatives may comprise at least one variable region (either a heavy chain or light chain variable region).

Other examples of suitable antibody derivatives and fragments include, without limitation, antibodies with poly-epitopic specificity, bispecific antibodies, multi-specific antibodies, diabodies, single-chain molecules, as well as FAb, F(Ab′)₂, Fd, Fab_{c}, and Fv molecules, single chain (Sc) antibodies, single chain Fv antibodies (scFv), individual antibody light chains, individual antibody heavy chains, fusions between antibody chains and other molecules, heavy chain monomers or timers, light chain monomers or timers, timers consisting of one heavy and one light chain, and other multimers. Single chain Fv antibodies may be multi valent. All antibody isotypes may be used to produce antibody derivatives, fragments, and portions. Antibody derivatives, fragments, and/or portions may be recombinantly produced and expressed by any cell type, prokaryotic or eukaryotic.

In a full-length antibody, cach heavy chain comprises a heavy chain variable region (abbreviated herein as HCVR or VH) and a heavy chain constant region. The heavy chain constant region comprises of three domains, CH1, CH2 and CH3. Each light chain comprises a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FWR or FR). Each VH and VL is composed of three CDRs and four FWRs, arranged from amino-terminus to carboxy-terminus in the following order: FWR1, CDR1, FWR2, CDR2, FWR3, CDR3, FWR4. Typically, the antigen binding properties of an antibody are less likely to be disturbed by changes to FWR sequences than by changes to the CDR sequences. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The antibodies may be derived from any species. For example, the antibodies may be mouse, rat, goat, horse, swine, bovine, camel, chicken, rabbit, donkey, llama, dromedary, shark, or human antibodies, as well as antibodies from any other animal species. For use in the treatment of humans, non-human derived antibodies may be structurally altered to be less antigenic upon administration to a human patient, including by chimerization or humanization or superhumanization.

As used herein, the terms “humanized,” “humanization,” and the like, refer to grafting of the murine monoclonal antibody CDRs disclosed herein to human FRs and constant regions. Also encompassed by these terms are possible further modifications to the murine CDRs, and human FRs, by the methods disclosed in, for example, Kashmiri et al. (2005) Methods 36(1):25-34 and Hou et al. (2008) J. Biochem. 144(1):115-120, respectively, to improve various antibody properties, as discussed below. Also see, for example, U.S. Patent Publication No. 2005/0033031; 2004/0086979; 2015/0038370; 2005/0226883; and 2021/0009666.

As used herein, the term “humanized antibodies” refers to mAbs and antigen binding fragments thereof, including antibody compounds, that have binding and functional properties similar to those disclosed herein, and that have FRs and constant regions that are substantially human or fully human surrounding CDRs derived from a non-human antibody.

As used herein, the term “FR” or “framework sequence” refers to any one of FRs 1 to 4. Humanized antibodies and antigen binding fragments encompassed by the present disclosure include molecules wherein any one or more of FRs 1 to 4 is substantially or fully human, i.e., wherein any of the possible combinations of individual substantially or fully human FRs 1 to 4, is present. For example, this includes molecules in which FR1 and 1-R2, FR1 and FR3, 1-R1, FR2, and FR3, etc., are substantially or fully human Substantially human frameworks are those that have at least 80% sequence identity to a known human germline framework sequence. Preferably, the substantially human frameworks have at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity, to a framework sequence disclosed herein, or to a known human germline framework sequence.

Fully human frameworks are those that are identical to a known human germline framework sequence. Human FR germline sequences can be obtained from the international ImMunoGeneTics (IMGT) database and from The Immunoglobulin FactsBook by Marie-Paule Lefranc and Gerard Lefranc, Academic Press, 2001, the contents of which are herein incorporated by reference in their entirety.

The Immunoglobulin Facts Book is a compendium of the human germline immunoglobulin genes that are used to create the human antibody repertoire, and includes entries for 203 genes and 459 alleles, with a total of 837 displayed sequences. The individual entries comprise all the human immunoglobulin constant genes, and germline variable, diversity, and joining genes that have at least one functional or open reading frame allele, and which are localized in the three major loci. For example, germline light chain FRs can be selected from the group consisting of: IGKV3D-20, IGKV2-30, IGKV2-29, IGKV2-28, IGKV1-27, IGKV3-20, IGKV1-17, IGKV1-16, 1-6, IGKV1-5, IGKV1-12, IGKVID-16, IGKV2D-28, IGKV2D-29, IGKV3-11, IGKV1-9, IGKV1-39, IGKVID-39, IGKVID-33, and IGKJ1-5; and germline heavy chain FRs can be selected from the group consisting of: IGHV1-2, IGHV1-18, IGHV1-46, IGHV1-69, IGHV2-5, IGHV2-26, IGHV2-70, IGHV1-3, IGHV1-8, IGHV3-9, IGHV3-11, IGHV3-15, IGHV3-20, IGHV3-66, IGHV3-72, IGHV3-74, IGHV4-31, IGHV3-21, IGHV3-23, IGHV3-30, IGHV3-48, IGHV4-39, IGHV4-59, IGHV5-51, and IGHJ 1-6.

Substantially human FRs are those that have at least 80% sequence identity to a known human germline FR sequence. Preferably, the substantially human frameworks have at least 85%, at least 90%, at least 95%, at least 96%, at least 97%, at least 98%, or at least 99% sequence identity, to a framework sequence disclosed herein, or to a known human germline framework sequence.

CDRs encompassed by the present disclosure include not only those specifically disclosed herein, but also CDR sequences having sequence identities of at least 80%, at least 85%, at least 90%, at least 95%, at least 96%, at least 97%, at least 98%, or at least 99% sequence identity to a CDR sequence disclosed herein. Alternatively, CDRs encompassed by the present disclosure include not only those specifically disclosed herein, but also CDR sequences having 1, 2, 3, 4, or 5 amino acid changes at corresponding positions compared to CDR sequences disclosed herein. Such sequence identical, or amino acid modified, CDRs preferably bind to the antigen recognized by the intact antibody.

Humanized antibodies in addition to those disclosed herein exhibiting similar functional properties according to the present disclosure can be generated using several different methods, including those disclosed by Almagro et al. (Frontiers in Biosciences. Humanization of antibodies. (2008) Jan. 1; 13:1619-33).

In one approach, the parent antibody compound CDRs are grafted into a human framework that has a high sequence identity with the parent antibody compound framework. The sequence identity of the new framework will generally be at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identical to the sequence of the corresponding framework in the parent antibody compound. In the case of frameworks having fewer than 100 amino acid residues, one, two, three, four, five, six, seven, eight, nine, or ten amino acid residues can be changed. This grafting may result in a reduction in binding affinity compared to that of the parent antibody. If this is the case, the framework can be back-mutated to the parent framework at certain positions based on specific criteria disclosed by Queen et al. (1991) Proc. Natl. Acad. Sci. USA 88:2869. Additional references describing methods useful to generate humanized variants based on homology and back mutations include as described in Olimpieri et al. (Bioinformatics 2015 Feb. 1; 31(3):434-435) and U.S. Pat. Nos. 4,816,397; 5,225,539; and 5,693,761; and the method of Winter and co-workers (Jones et al. (1986) Nature 321:522-525; Riechmann et al. (1988) Nature 332:323-327; and Verhocyen et al. (1988) Science 239:1534-1536).

Humanization began with chimerization, a method developed during the first half of the 1980's (Morrison, S. L., M. J. Johnson, L. A. Herzenberg & V. T. Oi: Chimeric human antibody molecules: mouse antigen-binding domains with human constant region domains. Proc. Natl. Acad. Sci. USA 81, 6851-5 (1984)), consisting of combining the variable (V) domains of murine antibodies with human constant (C) domains to generate molecules with ˜70% of human content.

Several different methods can be used to generate humanized antibodies, which are described herein. In one approach, the parent antibody compound CDRs are grafted into a human FR that has a high sequence identity with the parent antibody compound framework. The sequence identity of the new FR will generally be at least 60%, at least 65%, at least 70%, at least 75%, at least 80%, at least 85%, at least 90%, at least 95%, at least 96%, at least 97%, at least 98%, or at least 99% identical to the sequence of the corresponding FR in the parent antibody compound. In the case of FRs having fewer than 100 amino acid residues, one, two, three, four, five, or more amino acid residues can be changed. This grafting may result in a reduction in binding affinity compared to that of the parent antibody. If this is the case, the FR can be back mutated to the parent framework at certain positions based on specific criteria disclosed by Queen et al. (1991) Proc. Natl. Acad. Sci. USA 88:2869.

The identification of residues to consider for back-mutation can be carried out as described below. When an amino acid falls under the following category, the framework amino acid of the human germ-line sequence that is being used (the “acceptor FR”) is replaced by a framework amino acid from a framework of the parent antibody compound (the “donor FR”):
(a) the amino acid in the human FR of the acceptor framework is unusual for human frameworks at that position, whereas the corresponding amino acid in the donor immunoglobulin is typical for human frameworks at that position;
(b) the position of the amino acid is immediately adjacent to one of the CDRs; or
(c) any side chain atom of a framework amino acid is within about 5-6 angstroms (center-to-center) of any atom of a CDR amino acid in a three dimensional immunoglobulin model.

When each of the amino acids in the human FR of the acceptor framework and a corresponding amino acid in the donor framework is generally unusual for human frameworks at that position, such amino acid can be replaced by an amino acid typical for human frameworks at that position. This back-mutation criterion enables one to recover the activity of the parent antibody compound.

Another approach to generating humanized antibodies exhibiting similar functional properties to the antibody compounds disclosed herein involves randomly mutating amino acids within the grafted CDRs without changing the framework, and screening the resultant molecules for binding affinity and other functional properties that are as good as, or better than, those of the parent antibody compounds. Single mutations can also be introduced at cach amino acid position within each CDR, followed by assessing the effects of such mutations on binding affinity and other functional properties. Single mutations producing improved properties can be combined to assess their effects in combination with one another.

Further, a combination of both of the foregoing approaches is possible. After CDR grafting, one can back-mutate specific 1Rs in addition to introducing amino acid changes in the CDRs. This methodology is described in Wu et al. (1999, J. Mol. Biol. 294:151-162).

Applying the teachings of the present disclosure, a person skilled in the art can use common techniques, e.g., site-directed mutagenesis, to substitute amino acids within the presently disclosed CDR and FR sequences and thereby generate further variable region amino acid sequences derived from the present sequences. Up to all naturally occurring amino acids can be introduced at a specific substitution site. The methods disclosed herein can then be used to screen these additional variable region amino acid sequences to identify sequences having the indicated in vivo functions. In this way, further sequences suitable for preparing humanized antibodies and antigen-binding portions thereof in accordance with the present disclosure can be identified. Preferably, amino acid substitution within the frameworks is restricted to one, two, three, four, or five positions within any one or more of the four light chain and/or heavy chain FRs disclosed herein. Preferably, amino acid substitution within the CDRs is restricted to one, two, three, four, or five positions within any one or more of the three light chain and/or heavy chain CDRs. Combinations of the various changes within these FRs and CDRs described above are also possible.

That the functional properties of the antibody compounds generated by introducing the amino acid modifications discussed above conform to those exhibited by the specific molecules disclosed herein can be confirmed by the methods in Examples disclosed herein.

As described above, to circumvent the problem of eliciting human anti-murine antibody (HAMA) response in patients, murine antibodies have been genetically manipulated to progressively replace their murine content with the amino acid residues present in their human counterparts by grafting their complementarity determining regions (CDRs) onto the variable light (VL) and variable heavy (VH) frameworks of human immunoglobulin molecules, while retaining those murine framework residues deemed essential for the integrity of the antigen-combining site. However, the xenogeneic CDRs of the humanized antibodies may evoke anti-idiotypic (anti-Id) response in patients.

To minimize the anti-Id response, a procedure to humanize xenogeneic antibodies by grafting onto the human frameworks only the CDR residues most crucial in the antibody-ligand interaction, called “SDR grafting”, has been developed, wherein only the crucial specificity determining residues (SDRs) of CDRS are grafted onto the human frameworks. This procedure, described in Kashmiri et al. (2005, Methods 36(1):25-34), involves identification of SDRs through the help of a database of the three-dimensional structures of the antigen-antibody complexes of known structures, or by mutational analysis of the antibody-combining site. An alternative approach to humanization involving retention of more CDR residues is based on grafting of the ‘abbreviated’ CDRs, the stretches of CDR residues that include all the SDRs. Kashmiri et al. also discloses a procedure to assess the reactivity of humanized antibodies to sera from patients who had been administered the murine antibody.

Another strategy for constructing human antibody variants with improved immunogenic properties is disclosed in Hou et al. (2008, J. Biochem. 144(1):115-120). These authors developed a humanized antibody from 4C8, a murine anti-human CD34 monoclonal antibody, by CDR grafting using a molecular model of 4C8 built by computer-assisted homology modelling. Using this molecular model, the authors identified FR residues of potential importance in antigen binding. A humanized version of 4C8 was generated by transferring these key murine FR residues onto a human antibody framework that was selected based on homology to the murine antibody FR, together with the murine CDR residues. The resulting humanized antibody was shown to possess antigen-binding affinity and specificity similar to that of the original murine antibody, suggesting that it might be an alternative to murine anti-CD34 antibodies routinely used clinically.

Embodiments of the present disclosure encompass antibodies created to avoid recognition by the human immune system containing CDRs disclosed herein in any combinatorial form such that contemplated mAbs can contain the set of CDRs from a single murine mAb disclosed herein, or light and heavy chains containing sets of CDRs comprising individual CDRs derived from two or three of the disclosed murine mAbs. Such mAbs can be created by standard techniques of molecular biology and screened for desired activities using assays described herein. In this way, the disclosure provides a “mix and match” approach to create novel mAbs comprising a mixture of CDRs from the disclosed murine mAbs to achieve new, or improved, therapeutic activities.

Monoclonal antibodies or antigen-binding fragments thereof encompassed by the present disclosure that “compete” with the molecules disclosed herein are those that bind target antigens at site(s) that are identical to, or overlapping with, the site(s) at which the present molecules bind. Competing monoclonal antibodies or antigen-binding fragments thereof can be identified, for example, via an antibody competition assay. For example, a sample of purified or partially purified target antigen can be bound to a solid support. Then, an antibody compound, or antigen binding fragment thereof, of the present disclosure and a monoclonal antibody or antigen-binding fragment thereof suspected of being able to compete with such disclosure antibody compound are added. One of the two molecules is labelled. If the labelled compound and the unlabeled compound bind to separate and discrete sites on target antigen, the labelled compound will bind to the same level whether or not the suspected competing compound is present. However, if the sites of interaction are identical or overlapping, the unlabeled compound will compete, and the amount of labelled compound bound to the antigen will be lowered. If the unlabeled compound is present in excess, very little, if any, labelled compound will bind.

For purposes of the present disclosure, competing monoclonal antibodies or antigen-binding fragments thereof are those that decrease the binding of the present antibody compounds to the target antigen by about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, or about 99%. Details of procedures for carrying out such competition assays are well known in the art and can be found, for example, in Harlow and Lane (1988) Antibodies, A Laboratory Manual, Cold Spring Harbor Laboratory Press, Cold Spring Harbor, N.Y. Such assays can be made quantitative by using purified antibodies. A standard curve is established by titrating one antibody against itself, i.e., the same antibody is used for both the label and the competitor. The capacity of an unlabeled competing monoclonal antibody or antigen-binding fragment thereof to inhibit the binding of the labelled molecule to the plate is titrated. The results are plotted, and the concentrations necessary to achieve the desired degree of binding inhibition are compared.

Whether mAbs or antigen-binding fragments thereof that compete with antibody compounds of the present disclosure in such competition assays possess the same or similar functional properties of the present antibody compounds can be determined via these methods in conjunction with the methods described in Examples below. In various embodiments, competing antibodies for use in the therapeutic methods encompassed herein possess biological activities as described herein in the range of from about 50% to about 100% or about 125%, or more, compared to that of the antibody compounds disclosed herein. In some embodiments, competing antibodies possess about 50%, about 60%, about 70%, about 80%, about 85%, about 90%, about 91%, about 92%, about 93%, about 94%, about 95%, about 96%, about 97%, about 98%, about 99%, or identical biological activity compared to that of the antibody compounds disclosed herein as determined by the methods disclosed in the Examples presented below.

The mAbs or antigen-binding fragments thereof or competing antibodies useful in the compositions and methods can be any of the isotypes described herein. Furthermore, any of these isotypes can comprise further amino acid modifications as follows.

The antibodies may be minibodies. Minibodies comprise small versions of whole antibodies, which encode in a single chain the essential elements of a whole antibody. For example, the minibody may be comprised of the VH and VL domains of a native antibody fused to the hinge region and CH3 domain of an immunoglobulin molecule.

In some embodiments, the antibody may comprise non-immunoglobulin derived protein frameworks. For example, reference may be made to (Ku & Schutz, Proc. Nat. Acad. Sci. USA 92:6552-6556, 1995) which describes a four-helix bundle protein cytochrome b562 having two loops randomized to create CDRs, which have been selected for antigen binding.

Natural sequence variations may exist among heavy and light chains and the genes encoding them, and therefore, persons having ordinary skill in the art would expect to find some level of variation within the amino acid sequences, or the genes encoding them, of the antibodies described and exemplified herein. These variants preferably maintain the unique binding properties (e.g., specificity and affinity) of the parent antibody. Such an expectation is due in part to the degeneracy of the genetic code, as well as to the known evolutionary success of conservative amino acid sequence variations, which do not appreciably alter the nature of the encoded protein. Accordingly, such variants and homologs are considered substantially the same as one another and are included within the scope of the disclosure. The antibodies thus include variants having single or multiple amino acid substitutions, deletions, additions, or replacements that retain the biological properties (e.g., binding specificity and binding affinity) of the parent antibodies. The variants are preferably conservative but may be non-conservative.

Amino acid positions assigned to CDRs and FWRs may be defined according to Kabat Sequences of Proteins of Immunological Interest, National Institutes of Health, Bethesda, Md., 1987 and 1991 (also referred to herein as the Kabat numbering system). In addition, the amino acid positions assigned to CDRs and FWRs may be defined according to the Enhanced Chothia Numbering Scheme (www.bioinfo.org.uk/mdex.html). The heavy chain constant region of an antibody can be defined by the EU numbering system (Edelman, GM et al. (1969)., Proc. NatI. Acad. USA, 63, 78-85).

According to the numbering system of Kabat, VH FWRs and CDRs may be positioned as follows: residues 1-30 (FWR1), 31-35 (CDR1), 36-49 (FWR2), 50-65 (CDR2), 6694 (FWR3), 95-102 (CDR3) and 103-113 (FWR4), and VL FWRs and CDRs are positioned as follows: residues 1-23 (FWR1), 24-34 (CDR1), 35-49 (FWR2), 50-56 (CDR2), 57-88 (FWR3), 89-97 (CDR3) and 98-107 (FWR4). In some instances, variable regions may increase in length and according to the Kabat numbering system some amino acids may be designated by a number followed by a letter. This specification is not limited to FWRs and CDRs as defined by the Kabat numbering system, but includes all numbering systems, including the canonical numbering system or of Chothia et al. (1987) J. Mol. Biol. 196:901-17; Chothia et al. (1989) Nature 342:877-83; and/or Al-Lazikani et al. (1997) J. Mol. Biol. 273:927-48; the numbering system of Honnegher et al. (2001) J. Mol. Biol., 309:657-70; or the IMGT system discussed in Giudicelli et al., (1997) Nucleic Acids Res. 25:206-11. In some embodiments, the CDRs are defined according to the Kabat numbering system.

In some preferred embodiments, a fusion protein may comprise i) an antibody scaffold protein comprising a full-length antibody or a Fab fragment; and ii) a propeptide of procathepsin S; wherein the propeptide of procathepsin S is grafted into one or more complementarity determining regions (CDR) of the full-length antibody, or the propeptide of procathepsin S is grafted onto an N-terminus or C-terminus of a light chain and/or a heavy chain of the Fab fragment.

In other embodiments, the propeptide is a propeptide of procathepsin A, procathepsin B, procathepsin C, procathepsin D, procathepsin E, procathepsin F, procathepsin G, procathepsin H, procathepsin K, procathepsin L1, procathepsin L2, procathepsin O, procathepsin S, procathepsin W, or procathepsin Z.

Preferably, the propeptide of procathepsin S comprises the amino acid sequence

(SEQ ID NO: 2)
QLHKDPTLDHHWHLWKKTYGKQYKEKNEEAVRRLIWEKNLKFVMLHNLE
HSMGMHSYDLGMNHLGDMTSEEVMSLMSSLRVPSQWQRNITYKSNPNR.

In some embodiments, the propeptide of procathepsin S is grafted onto an N-terminus or C-terminus of a light chain and/or a heavy chain of the Fab fragment. Preferably, the Fab fragment comprises a Fab fragment, a Fab′ fragment, or a F(ab′)₂ fragment.

In some embodiments the propeptide (e.g., the propeptide of procathepsin S) is grafted into CDR1, CDR2, or CDR3 of a full-length antibody. In some embodiments the propeptide of procathepsin S (SEQ ID NO: 2) is grafted into the CDR3 of a full-length antibody. Alternatively, in some embodiments, the propeptide of procathepsin S replaces the CDR3 of the full-length antibody.

In some embodiments, the full-length antibody comprises a humanized full-length antibody or the Fab fragment comprises a humanized Fab fragment.

In some embodiments, the antibody scaffold protein comprises one of the fully humanized anti-Her2 antibody described in European Patent Publication No. EP2540745, the monoclonal antibodies against HER2 antigens disclosed in U.S. Pat. No. 8,722,362; and Herceptin (Trastuzumab) as described in U.S. Pat. No. 5,821,337 and U.S. Patent Publication US 2020/0155701). In some embodiments, a light chain of the Herceptin comprises an amino acid sequence of:

(SEQ ID NO: 8)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, a heavy chain of the Herceptin comprises an amino acid sequence of:

(SEQ ID NO: 9)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some embodiments, the antibody scaffold protein comprises SYNAGIS® (Palivizumab) (U.S. Pat. Nos. 5,824,307 and 9,139,642) and/or fragments thereof that specifically bind to a RSV antigen. Fragments of SYNAGIS® may be generated by any technique known to those skilled in the art. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain. Preferably, the fragment also binds to a RSV antigen, more preferably to the same epitope as SYNAGIS®.

In some embodiments, a light chain of the SYNAGIS® comprises an amino acid sequence of:

(SEQ ID NO: 10)
DIQMTQSPSTLSASVGDRVTITCKCQLSVGYMHWYQQKPGKAPKLLIYD
TSKLASGVPSRFSGSGSGTEFTLTISSLQPDDFATYYCFQGSGYPFTFG
GGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC.

In some embodiments, a heavy chain of the SYNAGIS® comprises an amino acid sequence of:

(SEQ ID NO: 11)
QVTLRESGPALVKPTQTLTLTCTFSGFSLSTSGMSVGWIRQPPGKALEW
LADIWWDDKKDYNPSLKSRLTISKDTSKNQVVLKVTNMDPADTATYYCA
RSMITNWYFDVWGAGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHT

In some embodiments, the antibody scaffold protein comprises a full-length antibody, and the fusion protein further comprises a coiled-coil domain disposed at each of an N-terminus end and a C-terminus end of the propeptide of procathepsin S. Canonical coiled-coil domains generally consist of two or more a-helices in a parallel or antiparallel orientation that are wrapped around each other into regular left-handed supercoiled bundles. Coiled-coil domains generally provide structural rigidity owing to their regular meshing of side chains. The coiled-coil domain may be derived from any of the following: cartilage-oligomeric matrix protein (COMP), mannose-binding protein A, coiled-coil serine-rich protein 1, polypeptide release factor 2, SNAP-25, SNARE, Lac repressor or apolipoprotein E, or other coiled-coil domains such as described in U.S. Patent Publication No. 2016/0002356 (Christensen). Preferably, the coiled-coil domain comprises GGSGAKLAALKAKLAALK (SEQ ID NO: 3) or ELAALEAELAALEAGGSG (SEQ ID NO: 4).

In other embodiments, a fusion protein or cathepsin S inhibitors described herein also include one or more spacer or linker peptides positioned at one or more of an N-terminus end and a C-terminus end of the propeptide of procathepsin S. As used herein, a “linker” or “spacer” peptide refers to an amino acid sequence of two or more amino acids in length. The linker can consist of neutral polar or nonpolar amino acids. A linker can be, for example, 2 to 100 amino acids in length, such as between 2 and 50 amino acids in length, for example, 3, 5, 10, 15, 20, 25, 30, 35, 40, 45, or 50 amino acids in length. Exemplary spacer peptides comprise the amino acid sequence GGGGS (SEQ ID NO: 5) or GGSG (SEQ ID NO: 6).

In some embodiments, a humanized cathepsin S-antibody inhibitor comprises a fusion protein of Formula I: A-B-C-D-A, wherein: A is an antibody scaffold protein; B is a first coiled-coil domain; C is a propeptide of procathepsin S; and D is a second coiled-coil domain; wherein Formula I optionally includes one or more peptide linkers E between A and B of segment A-B-and/or between D and A of segment-D-A. In some embodiments, B comprises SEQ ID NO: 3, C comprises SEQ ID NO: 2, D comprises SEQ ID NO: 4, and E comprises SEQ ID NO: 5 or SEQ ID NO: 6.

In some embodiments, a humanized cathepsin S-antibody inhibitor comprises the amino acid sequence of GGSGAKLAALKAKLAALKCGGGGSIEGRQLHKDPTL DHHWHLWKKTYGKQYKEKNEEAVRRLIWEKNLKFVMLHNLEHSMGMHSYDLGM NHLGDMTSEEVMSLMSSLRVPSQWQRNITYKSNPNRGGGGSCELAALEAELAALEA GGSG (SEQ ID NO: 7) inserted into or replacing a CDR3 of a heavy chain full length antibody.

Exemplary embodiments of a fusion protein and/or cathepsin S-antibody inhibitor include:

(Her-HC-CTSSpp IgG LC)
(SEQ ID NO: 8)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Her-HC-CTSSpp IgG HC
(SEQ ID NO: 12)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
GGSGAKLAALKAKLAALKCGGGGSIEGRQLHKDPTLDHHWHLWKKTYGK
QYKEKNEEAVRRLIWEKNLKFVMLHNLEHSMGMHSYDLGMNHLGDMTSE
EVMSLMSSLRVPSQWQRNITYKSNPNRGGGGSCELAALEAELAALEAGG
SGDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYIC
NVNHKPSNTKVDKKVEPKSCDKTHTCPPCPAPPVAGPSVFLFPPKPKDT
LMISRTPEVTCVVVDVSHEDPEVKFNWYVDGVEVHNAKTKPREEQYNST
YRVVSVLTVLHQDWLNGKEYKCKVSNKGLPSSIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K
Syn-LC-CTSSpp Fab LC
(SEQ ID NO: 13)
QLHKDPTLDHHWHLWKKTYGKQYKEKNEEAVRRLIWEKNLKFVMLHNLE
HSMGMHSYDLGMNHLGDMTSEEVMSLMSSLRVPSQWQRNITYKSNPNRG
GGGSDIQMTQSPSTLSASVGDRVTITCKCQLSVGYMHWYQQKPGKAPKL
LIYDTSKLASGVPSRFSGSGSGTEFTLTISSLQPDDFATYYCFQGSGYP
FTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYA
CEVTHQGLSSPVTKSFNRGEC
Syn-LC-CTSSpp Fab HC
(SEQ ID NO: 11)
QVTLRESGPALVKPTQTLTLTCTFSGFSLSTSGMSVGWIRQPPGKALEW
LADIWWDDKKDYNPSLKSRLTISKDTSKNQVVLKVTNMDPADTATYYCA
RSMITNWYFDVWGAGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHT

In some embodiments, a fusion protein/cathepsin S inhibitor is about 85%, about 86%, about 87%, about 88%, about 89%, about 90%, about 91%, about 92%, about 93%, about 94%, about 95%, about 96%, about 97%, about 98%, or about 99% identical to SEQ ID NO: 8.

In some embodiments, a fusion protein/cathepsin S inhibitor is about 85%, about 86%, about 87%, about 88%, about 89%, about 90%, about 91%, about 92%, about 93%, about 94%, about 95%, about 96%, about 97%, about 98%, or about 99% identical to SEQ ID NO: 12.

In some embodiments, a fusion protein/cathepsin S inhibitor is about 85%, about 86%, about 87%, about 88%, about 89%, about 90%, about 91%, about 92%, about 93%, about 94%, about 95%, about 96%, about 97%, about 98%, or about 99% identical to SEQ ID NO: 13.

In some embodiments, a fusion protein/cathepsin S inhibitor is about 85%, about 86%, about 87%, about 88%, about 89%, about 90%, about 91%, about 92%, about 93%, about 94%, about 95%, about 96%, about 97%, about 98%, or about 99% identical to SEQ ID NO: 11.

The disclosure also provides for composition comprising a cathepsin S inhibitor and a pharmaceutically acceptable diluent, excipient, or carrier.

The disclosure also provides for methods of treating a disease comprising administering an effective amount of the fusion protein or the humanized cathepsin S inhibitor as described herein to a subject in need thereof, thereby inhibiting cathepsin S and treating the disease, wherein the disease comprises one or more of cancer, Alzheimer's disease, a cardiovascular disease, and an autoimmune disease. Alternatively, a method of treating a disease comprises administering an effective amount of the composition as described herein to a subject in need thereof, thereby inhibiting cathepsin S and treating the disease, wherein the disease comprises one or more of cancer, Alzheimer's disease, a cardiovascular disease, and an autoimmune disease.

Mammalian diseases associated with cathepsin S including Alzheimer's disease, atherosclerosis, chronic obstructive pulmonary disease, cancer, diabetes, obesity, dyslipidacmia, dysglycaemia, hypertension, and certain autoimmune disorders, including, but not limited to juvenile onset diabetes, multiple sclerosis, pemphigus vulgaris, Graves' disease, myasthenia gravis, systemic lupus erythemotasus, Sjogren's syndrome, rheumatoid arthritis and Hashimoto's thyroiditis; allergic disorders, including, but not limited to asthma; and allogenic immune responses, including, but not limited to, rejection of organ transplants or tissue grafts. Cathepsin S is implicated in many cancers including, but not limited to, Oral, breast, lung, gastric, pancreatic, non-Hodgkin's lymphoma, cervical, bladder, childhood acute lymphoblastic leukemia, colorectal, renal, liver, thyroid, and glioblastoma.

### Results and Discussion

While anti-CTSS antibodies could be generated through animal immunization or in vitro library-based panning, it remains challenging to identify monoclonal antibodies that are capable of directly engaging with the CTSS catalytic site to inhibit its protease activity. Given our previous successes in designing inhibitory antibodies of CTSB and CTSL by utilizing their propeptides and the fact that the propeptide of proCTSS is an endogenous inhibitor of CTSS (25,26), we envisioned that functionally grafting the propeptide of proCTSS onto humanized immunoglobulin scaffolds may lead to potent antibody inhibitors with high specificity for CTSS.

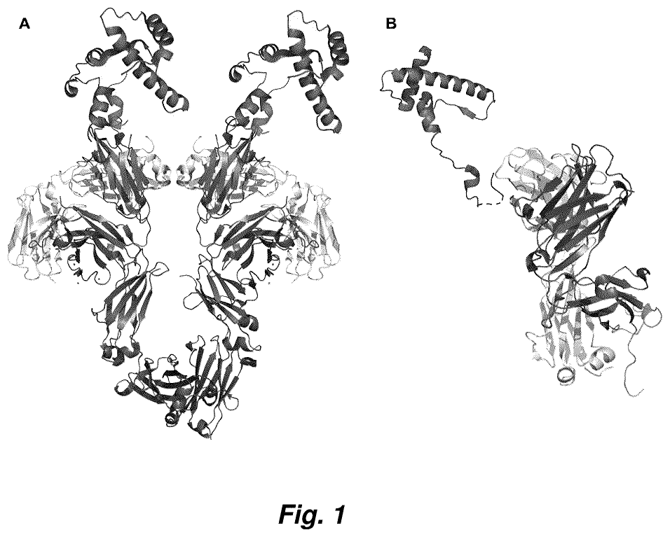

To this end, the propeptide of proCTSS (Q17-R113) was attempted for replacement of CDR3H (W99-M107) of the anti-HER2 antibody Herceptin (FIG. 1A) (30-32). To facilitate folding of the propeptide within the antibody variable region, a previously established coiled coil-based stalk motif that supports functional CDR fusions was utilized (17,18,21). In addition to the Herceptin IgG-based antibody inhibitor (Her-HC-CTSSpp IgG), we designed a Fab antibody inhibitor (Syn-LC-CTSSpp Fab) by fusing the propeptide to the N-terminus of the Fab light chain of the anti-RSV F protein antibody Synagis (FIG. 1B) (33,34). In comparison to the full-length antibody inhibitor, the Fab antibody inhibitor is much smaller and carries no fragment crystallizable (Fc) domain, possibly giving rise to unique physicochemical and pharmacological properties to meet distinct biomedical research needs (35). Unlike the Herceptin scaffold, Synagis is expected to recognize no human antigens and therefore allows for fusion of the propeptide at its light chain N-terminus, which may reduce impact of the fused propeptide on folding of the immunoglobulin.

The designed Herceptin full-length heavy chain CDR fusion and Synagis light chain N-terminal fusion were constructed by overlap extension PCR and cloned into mammalian expression vectors. In combination with the vector encoding Herceptin light chain or Synagis heavy chain Fab, the sequence-verified fusion vectors were used to express Her-HC-CTSSpp IgG and Syn-LC-CTSSpp Fab antibody fusions through transient transfection of Expi293F cells. Herceptin IgG and Synagis Fab antibodies were also expressed in the transiently transfected Expi293F cells by using the same type of expression vectors harboring native heavy and light chains.

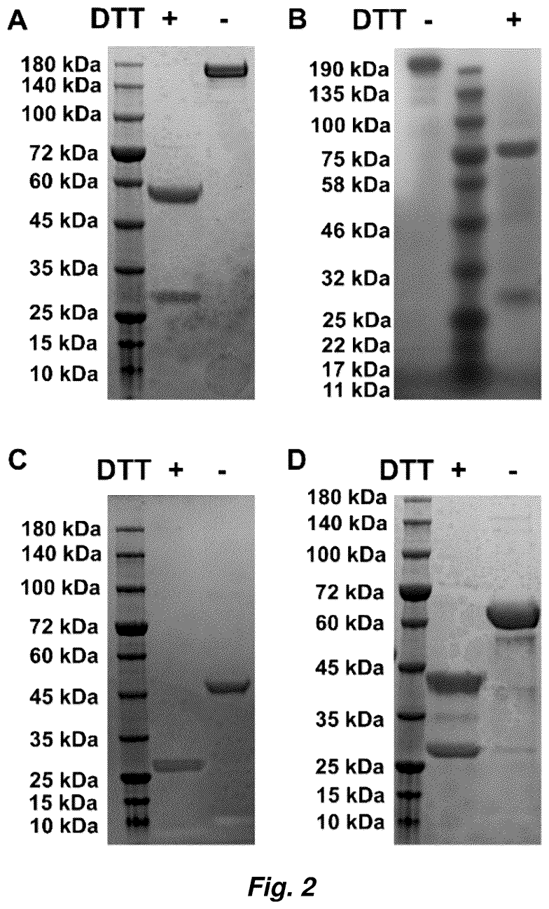

The expressed antibodies were purified by protein G affinity chromatograph. In comparison to the yield of Herceptin IgG (5.5 mg/L), the yield of Her-HC-CTSSpp IgG decreased to 0.6 mg/L, suggesting that the CDR3H fusion of propeptide reduces stability of Herceptin. Different from the IgG antibody CDR3H fusion, the Synagis Fab N-terminal fusion could be expressed in a yield (14.4 mg/L) comparable to that of native Synagis Fab (15 mg/L), indicating slightly minor effects of the fused propeptide on folding of the Synagis Fab antibody. SDS-PAGE gel analysis showed that the light chains of Herceptin IgG and Her-HC-CTSSpp IgG remained at the same position, whereas the heavy chain with the fused propeptide migrated at around 75 kDa, much higher than that of the Herceptin heavy chain and consistent with the design (FIG. 2A and B). Similarly, the light chain of Syn-LC-CTSSpp Fab shifted up to near 45 kDa relative to that of Synagis Fab on the basis of SDS-PAGE gels. And the heavy chains of both Fab antibodies migrated at the same molecular weight (FIG. 2C and D).

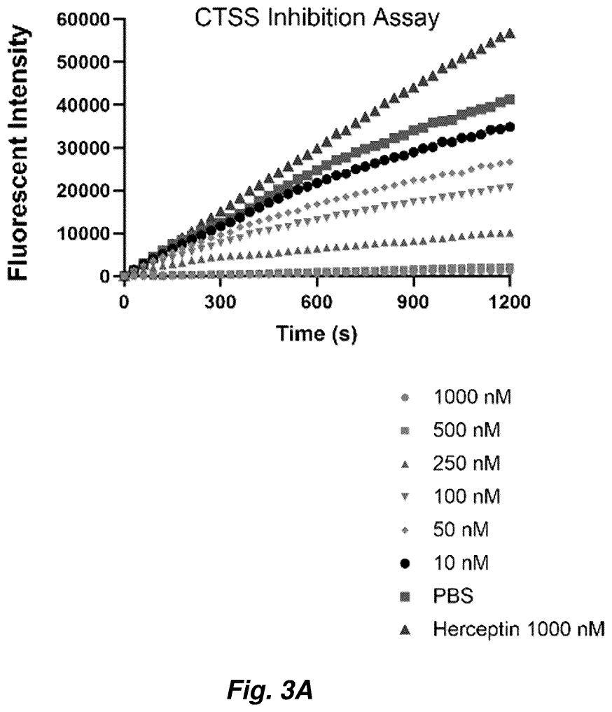

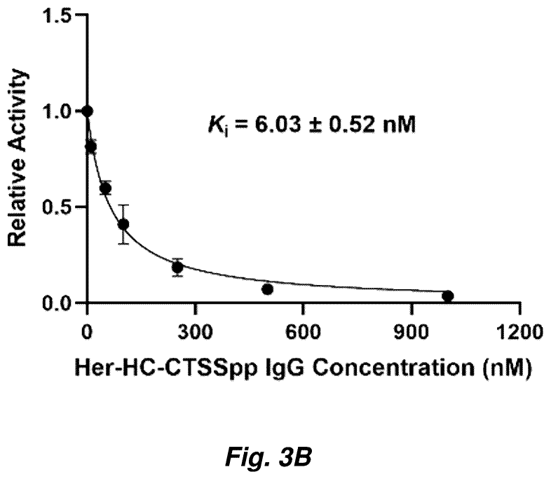

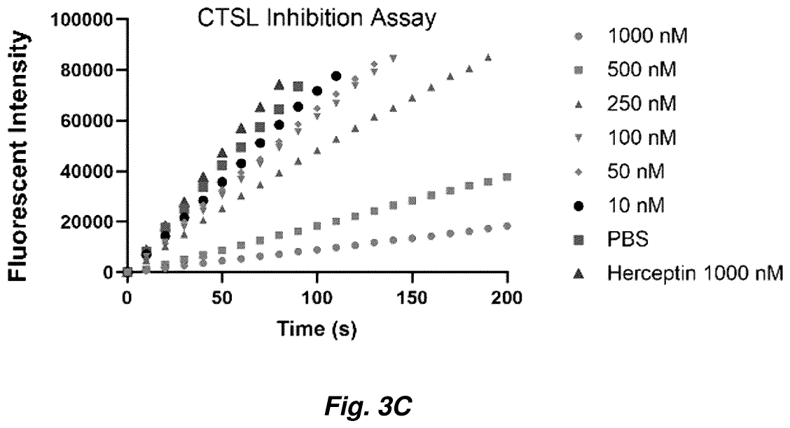

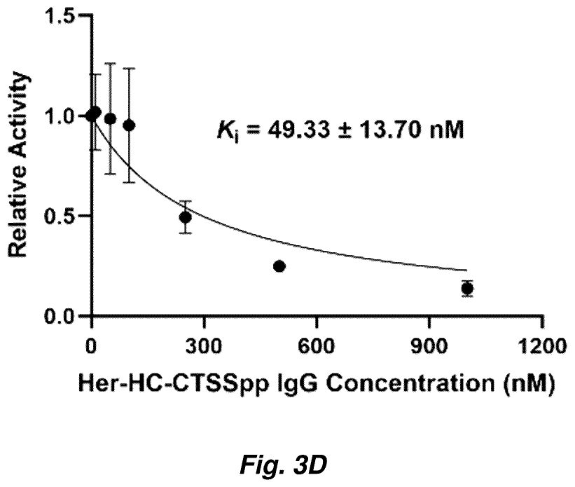

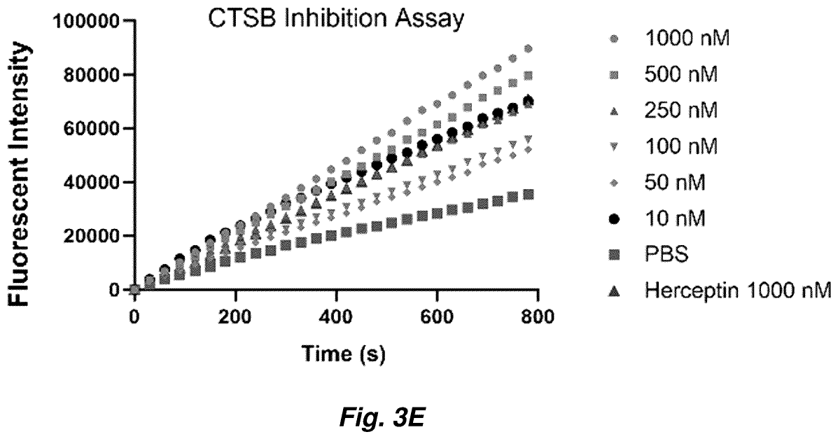

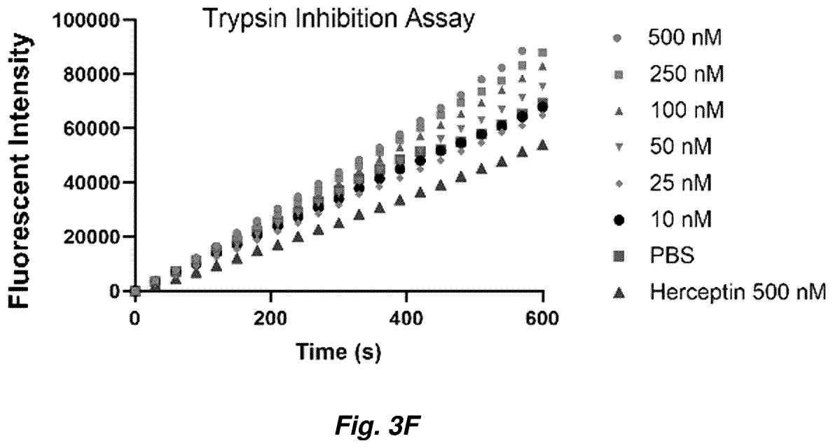

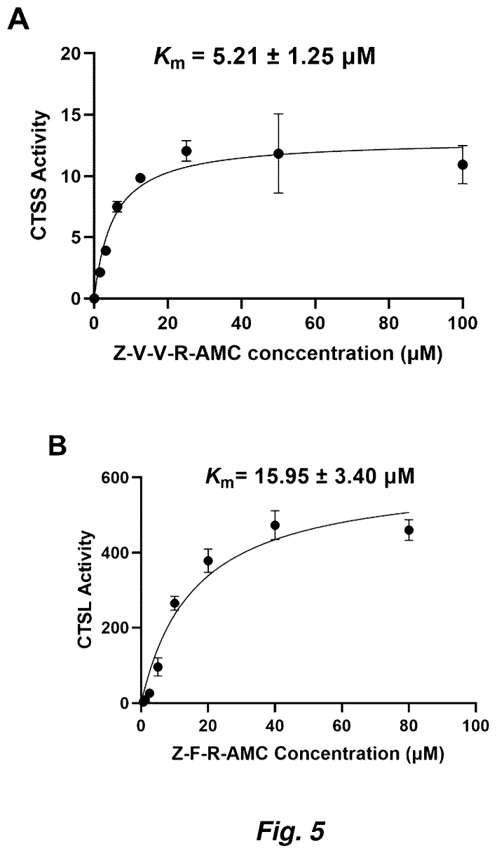

The inhibition activity of Her-HC-CTSSpp IgG was then examined with human CTSS through fluorescence-based activity assays using a fluorogenic peptide substrate Z-VVR-AMC (FIG. 5). Importantly, the designed Her-HC-CTSSpp IgG could potently inhibit human CTSS in a dose-dependent manner (FIG. 3A). By fitting the measured initial reaction rates into the competitive inhibition equation, the calculated Kᵢ of Her-HC-CTSSpp IgG for CTSS is 6.03±0.52 nM (FIG. 3B). CTSS and CTSL belong to the same subfamily of cathepsins. And the propeptide of proCTSS is a known inhibitor of CTSL (28,29). Therefore, the activity of Her-HC-CTSSpp IgG in inhibiting human CTSL was evaluated with a fluorogenic peptide substrate Z-FR-AMC (FIG. 5). The fluorescence-based signals indicated that human CTSL could be inhibited by Her-HC-CTSSpp IgG at nanomolar levels with a Kᵢ value of 49.33±13.70 nM (FIG. 3C and D). The parental antibody scaffold Herceptin displays no inhibition activity for human CTSS or CTSL at a concentration of 1000 nM. To further study inhibition specificity of Her-HC-CTSSpp IgG, fluorescence-based activity assays for human CTSB and bovine trypsin were carried out in the absence or presence of Her-HC-CTSSpp IgG. No inhibitory effects on CTSB or trypsin protease activity were observed for Her-HC-CTSSpp IgG at concentrations up to 1000 or 500 nM (FIG. 3E and F). These results demonstrate that grafting the propeptide of proCTSS into Herceptin CDR3H creates a potent and selective inhibitor for human CTSS.

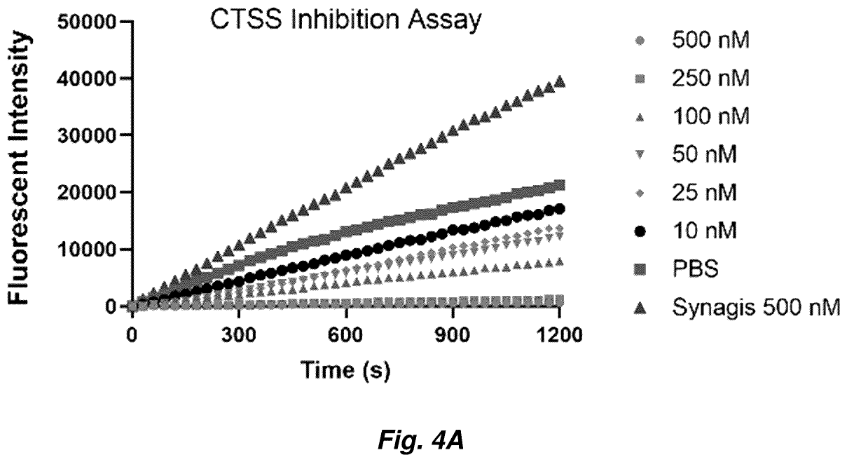

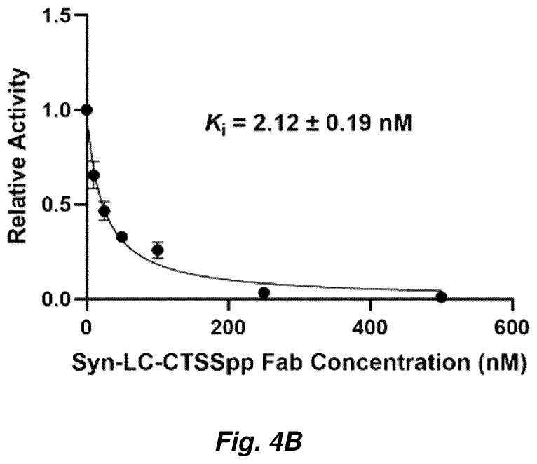

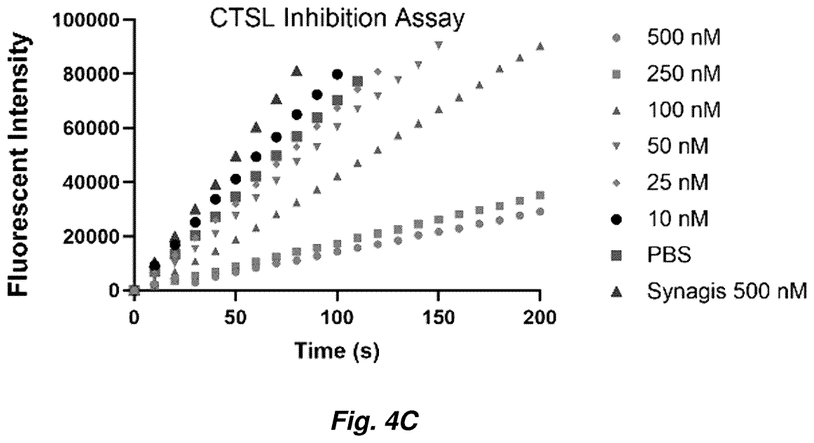

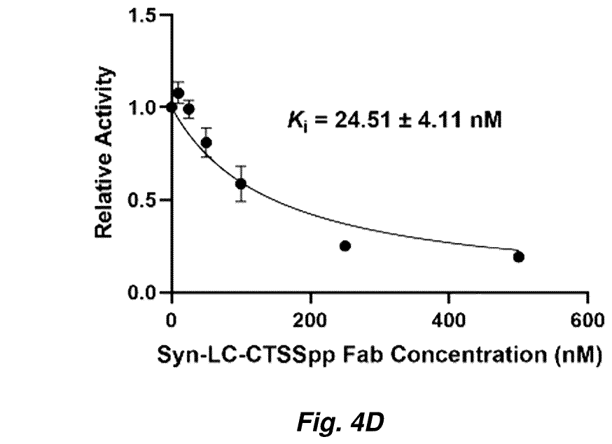

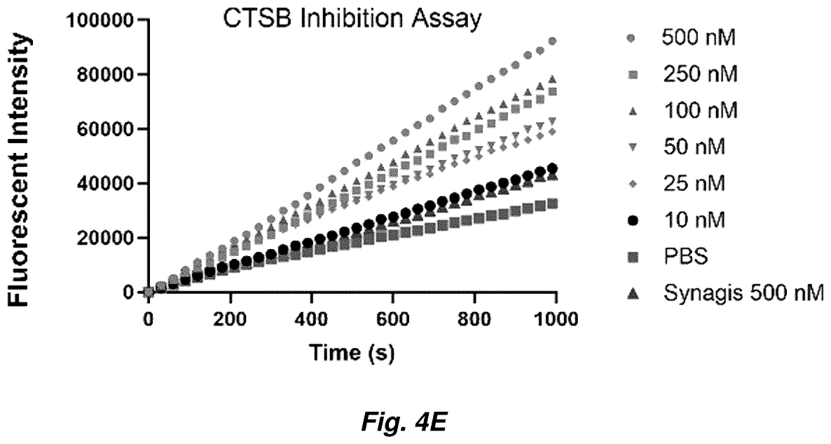

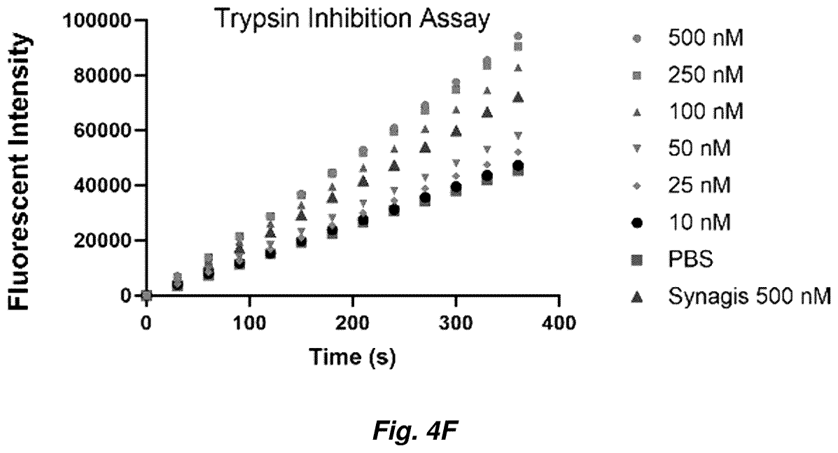

Next, inhibition potency and specificity of the designed Syn-LC-CTSSpp Fab antibody were analyzed by fluorescence-based proteolytic activity assays. Similar to Her-HC-CTSSpp IgG antibody, Syn-LC-CTSSpp Fab exhibits potent inhibitory activity toward human CTSS with a determined Kᵢ of 2.12±0.19 nM, slightly lower than that of Her-HC-CTSSpp IgG (FIG. 4A and B). Likewise, Syn-LC-CTSSpp Fab functions as a weaker antibody inhibitor against human CTSL. In comparison to CTSS, its potency for CTSL (Kᵢ: 24.51±4.11 nM) decreases by about 12-fold (FIG. 4C and D). Other inhibition specificity assays with human CTSB and bovine trypsin indicated that Syn-LC-CTSSpp Fab displays no inhibitory effects on these protease activities at concentrations up to 500 nM (FIG. 4E and F). As controls, Synagis Fab antibody at a concentration of 500 nM shows no inhibition activities for CTSS, CTSL, CTSB, or trypsin. Taken together, these results support the generation of a potent Fab antibody inhibitor with high specificity for human CTSS.

Guided by structure analysis, two different types of antibody inhibitors targeting human CTSS were successfully generated by genetically fusing the propeptide of proCTSS into immunoglobulin scaffolds. Both the full-length IgG and Fab antibody fusions could inhibit CTSS protease activity at low nanomolar levels. Their inhibition potency for human CTSS is 8-12-fold higher than that for human CTSL. Together with previous studies (25,26), this work establishes genetic fusion of cathepsin propeptide into immunoglobulins as a facile approach for developing inhibitory antibodies targeting cathepsins. In contrast to conventional library-based screening methods, utilizing the propeptides for rational design of antibody inhibitors is more efficient in identifying candidates with desired inhibition functions. Furthermore, the resulting propeptide-containing antibodies could be engineered by modulating adjacent interacting loops for enhanced affinity and/or specificity. In addition, bi-and multi-functional antibody inhibitors could be developed for targeting different types of proteases or diseased tissues. Future studies include measurements of in vitro and in vivo stability, improvement of potency and selectivity, and assessment of biological and pharmacological activity with cellular and animal models of CTSS-associated diseases.

### Conclusions

Humanized antibody inhibitors in both IgG and Fab formats for human CTSS were generated by engineering clinically approved antibodies with the propeptide of proCTSS. These antibody inhibitors reveal potent and selective activities against CTSS, providing new biologic agents for targeting CTSS proteolytic activity.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard-or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose, or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or acrosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, sec U.S. Pat. Nos. 4,992,478, 4,820,508, 4,608,392, and 4,559,157. Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, sec U.S. Pat. No. 4,938,949 (Borch). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Materials. All synthetic DNA fragments and polymerase chain reaction (PCR) primers were purchased from integrated DNA technologies (Coralville, IA). DNA restriction enzymes NheI (R3131S), EcoRI (R3101S), and T4 DNA ligase (M0202S) were purchased from New England Biolabs (Ipswich, MA). Recombinant human CTSS (1183-CY-010), human CTSL (952-CY-010), human CTSB (953-CY-010), and fluorogenic peptide substrate Z-FR-AMC (ES009) were purchased from R&D Systems (Minneapolis, MN). Fluorogenic peptide substrate Z-VVR-AMC (BML-P199-0010) and Boc-QAR-AMC (BML-P237-0005) werc purchased from Enzo Life Sciences (Farmingdale, NY). Bovine trypsin (T1426) and centrifugal filter units (UFC801008) were purchased from Sigma-Aldrich (St.Louis, MO). AccuPrime Pfx DNA polymerase (12344024), Zcocin (R25001), and Expi293F expression system (A14635) were purchased from Thermo-Fisher Scientific (Waltham, MA). Protein G resin (L00209), Tris-MOPS-SDS running buffer powder (M00138), and ExpressPlus-PAGE gels (M42015) were purchased from GenScript (Piscataway, NJ). DNA gel recovery kits (D4001), DNA clean & concentrator kits, and plasmid maxiprep kits (D4202) were purchased from Zymo Research (Irvine, CA).

Molecular cloning. The synthetic gene encoding the propeptide of proCTSS (Q17-R113) was purchased from integrated DNA Technologies and amplified by PCR with AccuPrime Pfx DNA polymerase with the forward and reverse primers. The amplified CTSS propeptide was confirmed by DNA gel electrophoresis and then purified by DNA gel extraction kits. To generate the Herceptin IgG-based antibody inhibitor (Her-HC-CTSSpp IgG), a flexible GGGGS (SEQ ID NO: 5) linker was added at each end of the propeptide. The entire sequence was then fused with the coiled coil-based stalk at each end in replacement of Herceptin CDR3H (W99-M107) via overlap extension PCR. The sequences of the coiled coil-based stalk are: H₂N-GGSGAKLAALKAKLAALK-COOH (SEQ ID NO: 3) in N-terminus and H₂N-ELAALEAELAALEAGGSG-COOH (SEQ ID NO: 4) in C-terminus. To create the Synagis Fab-based antibody inhibitor (Syn-LC-CTSSpp Fab), the amplified DNA fragment of propeptide was fused to the light chain N-terminus of Synagis with a GGGGS (SEQ ID NO: 4) linker by overlap extension PCR. The purified fusion genes were digested with restriction enzymes EcoRI and NheI and ligated into the pFuse vector backbone by T4 DNA ligasc. Electrocompetent DH10B cells were then transformed by the resulting ligation products via electroporation. The sequences of the resulting expression constructs were verified by DNA sequencing (Genewiz, CA).

Antibody expression and purification. Herceptin IgG, Her-HC-CTSSpp IgG, Synagis Fab, and Syn-LC-CTSSpp Fab were expressed through transient transfection of Expi293F cells with respective heavy and light chains. The Expi293F cells were grown at 37° C. with 5% CO₂ in the Expi293F expression medium. Culture media were harvested on day 5 post transfection and then centrifuged at 4,000 g for 30 minutes. The expressed antibodies were purified with protein G resins by cluting with 100 mM glycine (pH 2.7) and neutralizing with 1/10 volume of 1 M Tris-HCl (pH 8). The purified antibodies were buffer exchanged with PBS in centrifugal filters and analyzed by SDS-PAGE gels.

Michaelis constants of fluorogenic substrates. To determine Kₘ values of fluorogenic substrates Z-Val-Val-Arg-AMC (Z-VVR-AMC) and Z-Phe-Arg-AMC (Z-FR-AMC) for human CTSS and CTSL, respectively, Z-VVR-AMC and Z-FR-AMC were prepared as 10 mM stocks in DMSO. Following dilution with assay buffers (CTSS: 50 mM NaOAc, 250 mM NaCl, 5 mM DTT, pH 5; CTSL: 400 mM NaOAc, 4 mM EDTA, 8 mM DTT, pH 5.5), Z-VVR-AMC or Z-FR-AMC at various concentrations was incubated with human CTSS (1 ng/μL) or CTSL (0.1 ng/μL) in assay buffer at 25° C. The fluorescence intensities were recorded by a Synergy HI Hybrid Multi-Mode reader for 10 minutes (excitation: 380 nm; emission: 460 nm). The initial rates of CTSS or CTSL proteolytic activity were analyzed to determine Kₘ of Z-VVR-AMC or Z-FR-AMC by GraphPad Prism 9 (San Diego, CA).

Enzyme inhibition assays. To measure inhibition activities against CTSS for Her-HC-CTSSpp IgG and Syn-LC-CTSSpp Fab, 25 μL of human CTSS with a working concentration of 1 ng/μL in assay buffer (50 mM NaOAc, 250 mM NaCl, 5 mM DTT, pH 5) was incubated with 25 μL of Her-HC-CTSSpp IgG or Syn-LC-CTSSpp Fab at various concentrations and followed by the additions of 50 μL of fluorogenic substrate Z-VVR-AMC at a concentration of 100 μM.

To determine inhibition activities toward CTSL for Her-HC-CTSSpp IgG and Syn-LC-CTSSpp Fab, human CTSL was first diluted into 40 μg/mL in assay buffer (400 mM NaOAc, 4 mM EDTA, 8 mM DTT, pH 5.5) and incubated on ice for 15 minutes. Human CTSL (25 μL at a working concentration of 0.2 ng/μL in assay buffer) was then added with 25 μL of Her-HC-CTSSpp IgG or Syn-LC-CTSSpp Fab at various concentrations and 50 μL of fluorogenic substrate Z-FR-AMC at a concentration of 160 μM.

To evaluate inhibition activities on CTSB for Her-HC-CTSSpp IgG and Syn-LC-CTSSpp Fab, human CTSB was diluted into 10 μg/mL in assay buffer (34 mM NaOAc, 60 mM acetic acid, 4 mM EDTA, 5 mM DTT, pH 5.5) and incubated at room temperature for 15 minutes. Human CTSB (25 μL at a working concentration of 0.1 ng/μL in assay buffer) was mixed with 25 μL of Her-HC-CTSSpp IgG or Syn-LC-CTSSpp Fab at various concentrations and 50 μL of fluorogenic substrate Z-FR-AMC at a concentration of 160 μM.

To assess inhibition activities with bovine trypsin for Her-HC-CTSSpp IgG and Syn-LC-CTSSpp Fab, bovine trypsin was dissolved with 1 mM HCl into 1 mg/mL and then prepared into 10 nM in assay buffer (PBS, pH 7.4). Bovine trypsin (25 μL at a working concentration of 10 nM in assay buffer) was incubated with 25 μL of Her-HC-CTSSpp IgG or Syn-LC-CTSSpp Fab at various concentrations and 50 μL of fluorogenic substrate BOC-QAR-AMC at a concentration of 60 μM.

The fluorescence intensities (excitation: 380 nm; emission: 460 nm) for above assays were measured by a Synergy H1 Hybrid reader at 25° C. The inhibition constant Kᵢ was calculated by fitting initial reaction rates and inhibitor concentrations to the competitive inhibition equation: (V′₀/V₀)=(Kₘ+[S])/(Kₘ+[S]+(Kₘ[I]/Kᵢ)), where V′₀ is the initial reaction rate with inhibitor, V₀ is the initial reaction rate without inhibitor, [I] is the inhibitor concentration, and [S] is the substrate concentration.

### Example 2. Pharmaceutical Dosage Forms

The following formulations illustrate representative pharmaceutical dosage forms that may be used for the therapeutic or prophylactic administration of a compound or composition generically or specifically described herein (hereinafter referred to as ‘Composition X’, i.e., the enzymatic inhibitor):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, U.S. Patent Publication Number 2019/0119403 to Zhang et al. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A fusion protein comprising:
i) an antibody scaffold protein comprising a full-length antibody or a Fab fragment; and
ii) a propeptide of procathepsin S;
wherein the propeptide of procathepsin S is grafted into one or more complementarity determining regions (CDR) of the full-length antibody, or the propeptide of procathepsin S is grafted onto an N-terminus or C-terminus of a light chain and/or a heavy chain of the Fab fragment.

2. The fusion protein of claim 1, wherein the propeptide of procathepsin S comprises the amino acid sequence set forth in SEQ ID NO: 2.

3. The fusion protein of claim 1, wherein the propeptide of procathepsin S is grafted onto an N-terminus or C-terminus of a light chain and/or a heavy chain of the Fab fragment.

4. The fusion protein of claim 1, wherein the Fab fragment comprises a Fab fragment, a Fab′ fragment, or a F(ab′)₂ fragment.

5. The fusion protein of claim 1, wherein the propeptide of procathepsin S is grafted into CDR3 of the full-length antibody.

6. The fusion protein of claim 5, wherein the propeptide of procathepsin S is grafted into and effectively replaces the CDR3 of the full-length antibody.

7. The fusion protein of claim 1, wherein the antibody scaffold protein comprises a full-length antibody, and the fusion protein further comprising a coiled-coil domain disposed at each of an N-terminus end and a C-terminus end of the propeptide of procathepsin S.

8. The fusion protein of claim 7 wherein the coiled-coil domain comprises the amino acid sequence GGSGAKLAALKAKLAALK (SEQ ID NO: 3) or the amino acid sequence ELAALEAELAALEAGGSG (SEQ ID NO: 4).

9. The fusion protein of claim 1, further comprising one or more spacer peptides positioned at one or more of an N-terminus end and a C-terminus end of the propeptide of procathepsin S.

10. The fusion protein of claim 9 wherein the one or more spacer peptides comprise the amino acid sequence GGGGS (SEQ ID NO: 5) or GGSG (SEQ ID NO: 6).

11. The fusion protein of claim 1, wherein the full-length antibody comprises a humanized full-length antibody; or
the Fab fragment comprises a humanized Fab fragment.

12. The fusion protein of claim 11, wherein the full-length antibody comprises a heavy chain and a light chain, wherein the light chain comprises an amino acid sequence as set forth in SEQ ID NO: 8; and/or
the heavy chain comprises an amino acid sequence as set forth in SEQ ID NO: 12.

13. The fusion protein of claim 11, wherein the Fab fragment comprises a heavy chain and a light chain, wherein the light chain comprises an amino acid sequence set forth in SEQ ID NO: 13; and/or
the heavy chain comprises an amino acid sequence as set forth in SEQ ID NO: 11.

14. A humanized cathepsin S-antibody inhibitor comprising a fusion protein of Formula I: A-B-C-D-A, wherein:
A is an antibody scaffold protein;
B is a first coiled-coil domain;
C is a propeptide of procathepsin S; and
D is a second coiled-coil domain;
wherein Formula I optionally includes one or more peptide linkers E between A and B of segment A-B- and between D and A of segment -D-A.

15. The humanized cathepsin S inhibitor of claim 14, wherein B comprises SEQ ID NO: 3, C comprises SEQ ID NO: 2, D comprises SEQ ID NO: 4, and E comprises SEQ ID NO: 5 or SEQ ID NO: 6.

16. The humanized cathepsin S-antibody inhibitor of claim 14, wherein the humanized cathepsin S-antibody inhibitor comprises a heavy chain as set forth in SEQ ID NO: 12.

17. A composition comprising the fusion protein of claim 1 and a pharmaceutically acceptable diluent, excipient, or carrier.

18. A method of treating a disease comprising administering an effective amount of the fusion protein of claim 1 to a subject in need thereof, thereby inhibiting cathepsin S and treating the disease, wherein the disease comprises one or more of cancer, Alzheimer's disease, a cardiovascular disease, and an autoimmune disease.


SEQUENCE LISTING (13 sequences)

SEQ ID NO: 1 - protein, 331 aa, Homo sapiens [sequence omitted]

SEQ ID NO: 2 - protein, 97 aa, Homo sapiens
QLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNR

SEQ ID NO: 3 - protein, 18 aa, synthetic construct
GGSGAKLAALKAKLAALK

SEQ ID NO: 4 - protein, 18 aa, synthetic construct
ELAALEAELAALEAGGSG

SEQ ID NO: 5 - protein, 5 aa, synthetic construct
GGGGS

SEQ ID NO: 6 - protein, 4 aa, synthetic construct
GGSG

SEQ ID NO: 7 - protein, 149 aa, synthetic construct
GGSGAKLAALKAKLAALKCGGGGSIEGRQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRGGGGSCELAALEAELAALEAGGSG

SEQ ID NO: 8 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Homo sapiens
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 228 aa, Homo sapiens
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 12 - protein, 589 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKCGGGGSIEGRQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRGGGGSCELAALEAELAALEAGGSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 315 aa, synthetic construct
QLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC